(19) United States
(12) Patent Application Publication
(10) Pub. No.: US 2016/0120443 A1
(43) Pub. Date: May 5, 2016

(54) Single Chamber Volume Measurement Apparatus and Methods of Making and Using the Same

(71) Applicant: Near MARGALIT, Westlake Village, CA (US)

(72) Inventor: Near MARGALIT, Westlake Village, CA (US)

(21) Appl. No.: 14/867,996

(22) Filed: Sep. 28, 2015

Related U.S. Application Data
(60) Provisional application No. 62/074,570, filed on Nov. 3, 2014.

(51) Int. Cl. A61B 5/107; A61B 5/00

(52) U.S. Cl. CPC A61B 5/1073; A61B 5/1079; A61B 5/4872; A61B 2562/0219; A61B 2562/12

(57) ABSTRACT

A single chamber system or apparatus configured to measure a volume of a person, animal, and/or object, and methods of manufacturing and using the same, are disclosed. The system/apparatus includes a chamber having a door thereon or affixed thereto, and a fixed volume therein when the door is closed; an oscillating membrane or diaphragm in a wall of the chamber; a pressure sensor and/or a pressure transducer configured to measure pressure fluctuations in the chamber; and an oscillation amplitude detector coupled to the oscillating membrane or diaphragm. The chamber generally has a volume sufficient to enclose the person, animal, or object therein. The volume of the chamber generally does not vary with relatively small pressure changes or fluctuations.

### CROSS REFERENCE TO RELATED APPLICATION

This application claims the benefit of U.S. Provisional Patent Application No. 62/074,570, filed on Nov. 3, 2014, incorporated herein by reference as if fully set forth herein.

### FIELD OF THE INVENTION

The present invention generally relates to the field of volume and/or pressure measurements of objects, fluids, people, and/or animals. More specifically, embodiments of the present invention pertain to a single chamber apparatus and/or system for measuring the volume and/or pressure of objects, fluids, people, and/or animals, and methods of making and using the same.

### DISCUSSION OF THE BACKGROUND

Analysis of body volume may be very useful in a body-conscious society to determine progress on reducing body fat and tracking cosmetic appearance. Weight measurement does not provide a complete picture of physical status due to the different densities of muscle versus fat cells. Two individuals may have the same weight and height, but have vastly different body volume and composition. Specific measurements, such as waist circumference are often used as a proxy to determine changes in size of a person and expected or estimated body fat percentage or composition. However, it may be useful and beneficial for an individual to be able to measure the entire volume of the body to determine the progress that exercise and/or diet may have on their body shape and volume. A critical aspect of tracking such performance is the ability to easily and quickly get the measurement one is tracking

A number of techniques are available on the market to help individuals determine their body composition. The simplest method is measurement of circumferences of specific body parts, such as the waist or arms. This provides some information, but does not necessarily provide a complete picture of an individual's body volume.

A more complete measurement of body volume can be achieved by a technique known as hydrostatic weighing. In hydrostatic weighing, an individual is submerged in an enclosed water-filled container. The volume of water displaced by the individual's body is equal to the volume of the individual's body. The water volume displaced can be measured with simple weighing, if the container is shaped regularly. Although the hydrostatic weighing may be relatively accurate, it requires a person to go underwater, which may be unpleasant and/or burdensome for individuals. As a result, hydrostatic weighing is not a practical alternative for daily monitoring of body volume.

Other techniques to determine body fat percentage, but not necessarily body volume, are available. For example, a skinfold measurement using calipers can measure specific skinfold thickness, and the thickness can be correlated to body fat percentage. Alternatively, a bioelectric impedance analysis is another method that estimates body fat. The bioelectric impedance technique uses a high frequency electric current through the body to measure the body's impedance. Since fat cells and muscle cells have different impedances, an estimate of body fat may be obtained. However, the bioelectric impedance technique is greatly affected by an individual's state of hydration, and generally is not very accurate.

Alternatively, measurements of air pressure and air displacement to measure volume (i.e., plethysmography) may be used to measure an individual body fat and/or volume. Typically, plethysmography has the potential to achieve the accuracy of hydrostatic weighing, without the inconvenience of the subject going into water. Although this general technique to determine volume has been discussed at least since the 1940s, there has yet to be a solution that is simultaneously accurate, fast, simple, and low cost.

In an ideal solution, obtaining volume measurements should be similar to obtaining weight using a scale. For example, an individual should be able to enter a chamber and obtain a volume measurement in a matter of seconds. Currently, a commercially available product, the Bod Pod body composition tracking system (Cosmed USA, Inc., Concord, Calif.), has not achieved a desired level of simplicity, speed, and cost. Generally, a medical assistant is necessary to operate the system as the person is being measured, and the overall measurement process may be relatively lengthy. In addition, during the standard and/or manufacturer's recommended measurement process, the individual is required to wear a very tight fitting bathing suit. The cost of using such system is relatively high for daily or frequent measurements. In addition, this body composition tracking system relies on two connected chambers in which an oscillating membrane creates a pressure variation between the two chambers. Volume is calculated by using the relative pressure measurements in the two chambers.

Conventional single chamber systems that use an oscillating membrane do not take into consideration amplitude changes and variations that may occur in the chamber when a subject is inside the chamber. Such a system is generally intended to monitor astronauts' health by providing rough estimations of body volume. However, such conventional systems fail to take into account the impact that a human and/or animal subject has on the pressure and/or impedance measurement using an oscillating membrane. The heat from the living subject and the variations in air pressure from the subject's lungs cause variations in the amplitude of the oscillating membrane. To overcome such challenges, several inventions use a dual chamber between the oscillating membranes. In such configurations, a second chamber is used to neutralize the impact of amplitude fluctuations. To calculate the volume, the pressure ratio between the two chambers is determined.

This “Discussion of the Background” section is provided for background information only. The statements in this “Discussion of the Background” are not an admission that the subject matter disclosed in this “Discussion of the Background” section constitutes prior art to the present disclosure, and no part of this “Discussion of the Background” section may be used as an admission that any part of this application, including this “Discussion of the Background” section, constitutes prior art to the present disclosure.

### SUMMARY OF THE INVENTION

The present invention relates to a system or apparatus, comprising a chamber having a door thereon or affixed thereto, and a fixed volume therein when the door is closed; an oscillating membrane or diaphragm in a wall of the chamber; a pressure sensor and/or a pressure transducer configured to measure pressure fluctuations in the chamber; and an oscillation amplitude detector coupled to the oscillating membrane or diaphragm. The system or apparatus may be a single chamber apparatus, and may be configured to measure a volume of a person, animal, and/or object. As a result, the chamber generally has a volume sufficient to enclose the person, animal, or object therein. In addition, the volume of the chamber generally does not vary with relatively small pressure changes or fluctuations.

In various embodiments, the system or apparatus further comprises a computer or logic configured to determine a pressure amplitude in the chamber resulting from oscillations of the oscillating membrane or diaphragm and a volume displacement from oscillation or position amplitudes of the oscillating membrane or diaphragm with and without a subject or object in the chamber. In some examples, the system or apparatus (or, more particularly, the computer or logic) is configured to calculate a percentage or other proportion of body fat of a person and/or animal (e.g., in the chamber). The computer or logic may be on the exterior of the apparatus, and can be further configured to control operations of the apparatus and perform calculations to determine the body volume, body fat content, and/or weight of the person or animal. Thus, the system or apparatus may further comprise a weight scale in the chamber.

In other embodiments of the system or apparatus, the chamber has a cylindrical shape. Alternatively, the chamber may have a shape and/or cross-section selected from the group consisting of spherical, semi-cylindrical, hexagonal, octagonal, pentagonal, semi-circular, and combinations thereof. To facilitate observation of the subject or object and increase safety, the chamber may have one or more transparent walls.

In further embodiments, the chamber comprises (i) a first section comprising the door and (ii) a second, stationary section configured to house or contain a subject or object. In addition, the chamber may comprise a latchable handle and/or a seal or molding configured to create an airtight seal between the door and the stationary section of the chamber. In one case, the handle is on an internal surface of the chamber. Alternatively, the handle can be on an external surface of the door. In further embodiments, the door may be secured by one or more magnetic strips on the surface of the door and/or the stationary section of the chamber in an area or region where the door and the stationary section of the chamber overlap. In such embodiments the other of the door and the stationary section may have one or more magnetic or ferromagnetic metal strips on the complementary surface.

In still further embodiments of the system or apparatus, the oscillating membrane or diaphragm may comprise a rigid oscillating membrane or diaphragm. For example, the rigid oscillating membrane or diaphragm may comprise a speaker, a plate, or a disc, and it may have an area of from 10 to 5000 cm². When the rigid oscillating membrane or diaphragm comprises the speaker, the oscillation amplitude detector may comprise first and second coils. The first coil is generally configured to drive or power the speaker, and the second coil is generally configured to generate a voltage in response to motion of the speaker. In various implementations, the oscillating membrane or diaphragm may oscillate at a frequency of from 1 to 100 Hz, or any value or range of values therein (e.g., from 1 to 30 Hz). The system or apparatus also generally further includes one or more wires connecting the oscillation amplitude detector to the computer or logic.

In even further embodiments of the system or apparatus, the oscillation amplitude detector may comprise a position sensing element, such as (i) an optical light source and a light detector or (ii) an accelerometer. The oscillation amplitude detector may be configured to measure movement and/or displacement of the oscillating membrane or diaphragm. In some examples, the oscillation amplitude detector outputs a signal or response proportional to an oscillation or position amplitude of the oscillating membrane or diaphragm. In some embodiments, the position sensing element may comprise a capacitive sensing device configured to measure movement and/or displacement of the oscillating membrane or diaphragm.

Another aspect of the present invention relates to a method of determining a volume of a person, animal, or object in a single chamber volume measuring apparatus, comprising measuring both a first pressure amplitude and a first oscillation (or position) amplitude of an oscillating membrane or diaphragm in the apparatus without the person, animal, or object therein; placing the person, animal, or object in the apparatus; measuring a second pressure amplitude and a second oscillation (or position) amplitude of the oscillating membrane or diaphragm with the person, animal, or object therein; and calculating the volume of the person, animal, or object. However, the sequence of measuring the first and second pressure amplitudes and oscillation (or position) amplitudes can be reversed (i.e., the method may comprise measuring the pressure amplitude and the membrane/diaphragm oscillation [or position] amplitude with the person, animal, or object in the chamber, then measuring the pressure amplitude and the membrane/diaphragm oscillation [or position] amplitude without the person, animal, or object in the chamber). In one embodiment, the method further comprises calculating a body fat percentage of the person or animal (e.g., the person).

In some embodiments, the method further comprises measuring a first pressure difference in the single chamber during oscillation of the oscillating membrane or diaphragm without the person, animal, or object therein, and measuring a second pressure difference in the single chamber during oscillation of the oscillating membrane or diaphragm with the person, animal, or object therein (or vice versa). Additionally or alternatively, the method may further comprise determining a volume of air in the single chamber with the person, animal, or object therein, and optionally, determining a difference between the known volume of the empty chamber and the volume of air in the chamber with the person, animal, or object therein. The method may also further comprise determining a weight of the person, animal, or object using a weight scale; determining a density of the person, animal, or object by dividing the weight of the person, animal, or object by the volume of the person, animal, or object; and/or converting the density of the person, animal, or object to a body fat percentage.

Another aspect of the present invention relates to a method of manufacturing a system or apparatus to measure a volume of a person, animal, or object, or body fat percentage of the person or animal, comprising attaching door to a chamber; mounting or positioning an oscillating membrane or diaphragm in a wall of the chamber or in the door; placing a pressure sensor/transducer on an internal surface of the chamber; and coupling an oscillation amplitude detector to a surface of the oscillating membrane or diaphragm. The method may further comprise placing, attaching, or affixing a weight scale in or to the chamber. In various embodiments, the chamber has rigid walls, one or more of the walls and/or the door are transparent, the chamber comprises a stationary section configured to house, contain, or hold the person, animal, or object, the chamber has a volume sufficient to enclose the person, animal or object.

In further embodiments, the method may further comprise mounting or connecting a computer or operational logic to the apparatus. In one example, the oscillation amplitude detector is configured to provide a signal to the computer or operational logic, the signal being proportional to a displacement or an oscillation or position amplitude of the membrane or diaphragm. The method may further comprise programming a memory in the computer or operational logic with a set of instructions to (i) measure a pressure difference during oscillations of the oscillating membrane or diaphragm before and after the person, animal, or object enters the chamber (or with and without the person, animal, or object in the chamber), (ii) determine a ratio of oscillation amplitudes of the oscillating membrane or diaphragm before and after the person, animal, or object enters the chamber, and (iii) determine a volume of air in the chamber displaced by the person, animal, or object using the oscillation amplitudes and pressure differences.

In other or further embodiments, the method may further comprise forming or attaching a seal or molding on a surface of at least one of the door and the chamber, and/or forming or attaching a latchable handle on an internal or external surface of the door or chamber. The seal or molding may be configured to make the chamber airtight when the door is closed and the pressure inside the chamber changes. Alternatively or additionally, the method may further comprise placing or affixing one or more magnetic strips on the surface of the door and/or the stationary section of the chamber in an area or region where the door and the stationary section of the chamber overlap, and optionally, placing or affixing one or more magnetic or ferromagnetic metal strips on the complementary surface of the other of the door and the stationary section. Thus, the force keeping the door closed may either be magnetic or mechanical in nature, so that the volume of the chamber does not change substantially with changes in internal chamber pressure relative to outside pressure.

### BRIEF DESCRIPTION OF THE DRAWINGS

FIG. 1 is a diagram showing an exemplary embodiment of a single chamber body volume measurement apparatus in accordance with the present invention.

FIG. 2 shows a surface of an exemplary oscillating mechanism in accordance with the present invention.

FIG. 3 is a flow chart showing an exemplary method of a making a body volume measurement in a single chamber apparatus in accordance with the present invention.

FIG. 4 is a flow chart showing an exemplary method of a manufacturing a single chamber a body volume measurement apparatus in accordance with the present invention.

### DETAILED DESCRIPTION

Reference will now be made in detail to various embodiments of the invention, examples of which are illustrated in the accompanying drawings. While the invention will be described in conjunction with the following embodiments, it will be understood that the descriptions are not intended to limit the invention to these embodiments. On the contrary, the invention is intended to cover alternatives, modifications and equivalents that may be included within the spirit and scope of the invention as defined by the appended claims. Furthermore, in the following detailed description, numerous specific details are set forth in order to provide a thorough understanding of the present invention. However, it will be readily apparent to one skilled in the art that the present invention may be practiced without these specific details. In other instances, well-known methods, procedures, components, and circuits have not been described in detail so as not to unnecessarily obscure aspects of the present invention.

The present invention concerns a method and apparatus for performing volume measurement of an object, person, and/or animal. The apparatus includes a single rigid chamber and an oscillating membrane or diaphragm. The present invention provides the accuracy of a dual chamber with the simplicity and cost advantages of a single chamber system. Thus, the present invention provides a cost effective, efficient, and practical system that achieves accurate volume measurements using air displacement.

The present invention greatly improves accuracy of volume measurements by adding an oscillation amplitude detector coupled to (e.g., on or in optical or electromagnetic communication with) a surface or an object on the surface of the oscillating membrane. To measure human, animal, and/or object volume with an accuracy sufficient to determine body fat percentage, the volume measurement should be extremely accurate (e.g., with accuracy, results and/or sensitivity better than 0.1% of the chamber volume). Without the oscillation amplitude detector (e.g., position sensing element) on and/or coupled to the oscillating rigid membrane, an accuracy sufficient for measuring human body fat percentage or composition may not be achieved with a single chamber. Thus, the present invention provides a commercially viable single chamber apparatus configured to accurately measure body fat percentage.

The present apparatus may be useful in medical offices, physical therapy and/or training facilities, and professional and amateur athletic training facilities for measuring the volume and/or calculating a percentage or other proportion of body fat of a person. The present apparatus may be useful in veterinary medicine, on a ranch or farm, or in animal husbandry for measuring the volume and/or calculating a percentage or other proportion of body fat of an animal. Also, certain objects may not be regularly shaped and/or submersible in water, and the present apparatus is useful for measuring such objects and fluids, among other objects and/or other uses.

The present invention provides a faster and more accurate apparatus and method to measure body volume, achieving new levels of performance that are convenient and cost effective. The present invention advantageously provides an apparatus that effectively, efficiently, and inexpensively measures a whole body volume by using a direct oscillation amplitude detector that directly measures the movement of the diaphragm. The direct position or amplitude measurement of the diaphragm is used for normalization, rather that using pressure in an alternate chamber to normalize the pressure in the subject chamber. Thus, the present apparatus and/or system may be smaller, cheaper, and more reliable than conventional systems.

Alternatively, various amplitude detecting (e.g., position sensing) technologies may be used to directly measure movement of the membrane or diaphragm. For example, an optical light source that irradiates a reflective surface on the diaphragm or membrane may be used in conjunction with a light sensor that can measure distance (e.g., within +/−1 mm, tenths of an mm, etc.). Another example includes a micro-electro-mechanical system (MEMS) accelerometer (e.g., chip) that may be used to translate acceleration of the diaphragm surface to detect the relative position of the diaphragm movement (e.g., the minimum and maximum positions of the diaphragm during oscillation). Other methods, such as capacitive sensing or introducing one or more inductive coils, may also be used. Alternatively and/or additionally, the oscillation amplitude may be indirectly measured using a laser. For example, a laser may be positioned to emit light (e.g., a laser beam) toward a mirror or other reflective surface positioned on the membrane or diaphragm. The laser beam reflects or bounces off the mirror, and the reflected laser beam is detected by a light detector (e.g., on the chamber wall) that detects or determines relative movement and/or positions of the membrane or diaphragm.

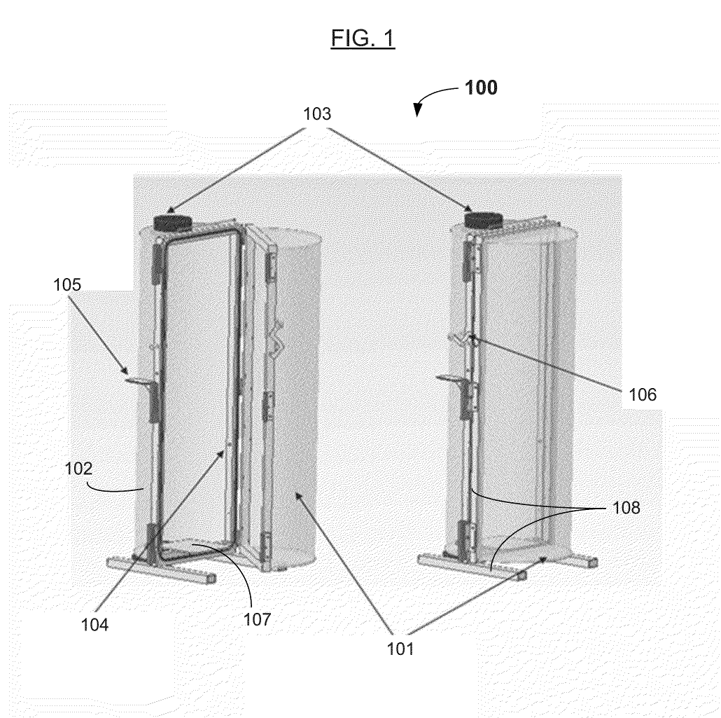

FIG. 1 is a diagram showing an exemplary embodiment of a single chamber body volume measurement apparatus 100 according to the present invention. The chamber may have a cylindrical shape that is split or divided in the middle section of the chamber, forming two major sections. The first major section is a moveable portion (e.g., a door) 101 configured to open and close the chamber 100, and the second major section is a stationary portion 102 in which the subject/object stands or is otherwise positioned. One or more walls and/or the door 101 of the chamber 100 (or portions thereof) may be transparent, and a metal frame 108 that supports the chamber may be present.

A subject enters and exits the cylinder shaped chamber 100 using the door 101. The chamber may have another shape (e.g., spherical) and/or cross-section (e.g., semi-cylindrical, hexagonal, octagonal, pentagonal, semi-circular, combinations thereof, etc.). The rigidity of the chamber generally improves with increasing roundness, so for a chamber of given volume and the same material(s), a cylindrical chamber is generally more rigid than a semi-cylindrical chamber. A spherical chamber (i.e., the most rigid shape) may be fitted with a horizontal, planar floor that is sealed to the chamber and (if necessary) to itself across the door opening, or alternatively, that has holes therein to permit air flow between the chamber spaces above and below the floor.

The height, width and depth of the exemplary chamber 100 as shown is constant and/or uniform across the chamber, but are not required to be so. However, the walls of the chamber 100 (including the door 101) are stiff and/or rigid (e.g., they do not noticeably bend under low pressure and/or slight vacuum). Closing the door 101 and sealing the door closed using a latchable handle 106 generally creates an airtight or semi-airtight seal between the sections of the chamber. The handle 106 may be on an external surface of the door 101 and/or an internal surface of the door 101, and latch onto a peg or other fixture 109. In addition, an airtight or substantially airtight seal or molding may be between the fixed section of chamber 100 and the door 101 (e.g., on the front edges or surface of the fixed section of the chamber 100 and/or around the periphery of the door 101). In one example, to increase accuracy of the volume displacement measurement, the seal or molding compresses by a known amount at one or more predetermined pressures in the chamber 100 when the door 101 is closed. If the surface of the sealing joint is mechanically well positioned, a rigid seal such as a magnetic strip can also be used to achieve a substantially airtight or semi-airtight seal of the chamber. Thus, the seal between the door 101 and the stationary part 102 of the chamber 100 may be soft (e.g., compressible) or hard (e.g., non-compressible).

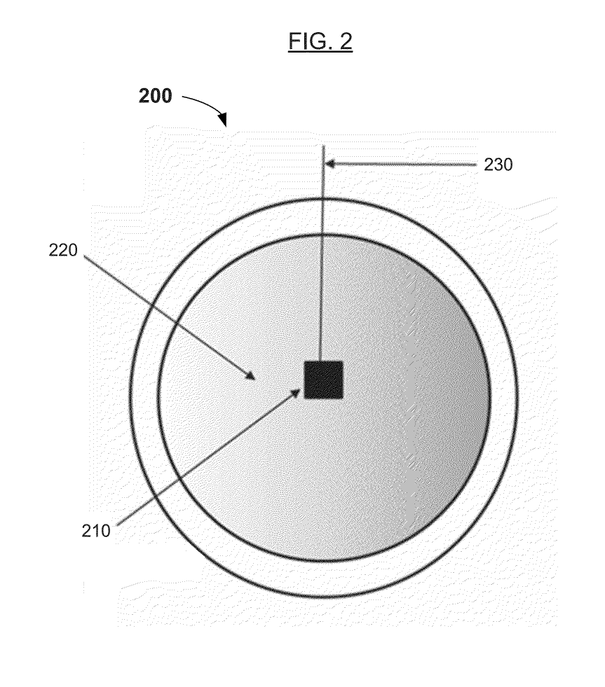

In exemplary embodiments of the present invention, the apparatus includes an oscillating membrane. For example, a subwoofer or other speaker, an oscillating diaphragm, or other similar device may be used. The oscillating membrane may be a rigid oscillating membrane, placed in an opening in a wall of the chamber. A rubber gasket placed around the edge of the membrane or diaphragm may hold the oscillating membrane or diaphragm in place. Alternatively or additionally, a silicone sealant or other similar sealing material is placed around the membrane and/or the rubber gasket (or similar structure) to seal the opening in the chamber wall. Alternatively, the oscillating membrane may be or comprise a rigid disc or plate having a corrugated peripheral region configured to enable the rigid disc or plate to oscillate. In various embodiments of the present invention, the apparatus includes an oscillation amplitude detector (see 210 of FIG. 2) configured to measure the movement and/or displacement of the oscillating membrane. The oscillating membrane and oscillation amplitude detector (e.g., position sensor) are discussed in further detail in FIG. 2.

A pressure sensor or a pressure transducer 104 may be read electronically, and the pressure readings from the chamber 100 may be stored in an electronic memory (e.g., in a desktop, laptop, tablet, or handheld computer, in a volatile and/or non-volatile memory electronically connected to a controller or other logic that controls operations of the apparatus 100, etc.). The pressure reading from pressure sensor 104 is the pressure in chamber 100, as the sensor is directly exposed or connected to the air in the chamber 101.

The display unit and/or computer 105 may be included on the exterior portion of the apparatus 100. Prior to entering the chamber, the subject may directly initiate a measurement and obtain measurement results, which may include body volume, weight, body fat content (e.g., as a percentage of body weight), etc. In addition, the display or computer 105 may control operations of the apparatus 100 and/or perform calculations necessary to determine body volume, body fat content, weight, etc. (e.g., performs calculations on the information stored in the memory, and displays messages, readings and/or the results on a display unit and/or computer 105). An optional weight scale 107 inside the chamber 100 allows for simultaneously measuring the subject's weight along with the subject's body volume. Weight and volume can be used to determine a person's or animal's density, which in turn can be used to determine body composition, such as fat percentage.

FIG. 2 shows a surface of an exemplary oscillating membrane 200 suitable for use in the present invention. In exemplary embodiments of the present invention, an oscillation amplitude (e.g., position sensing) detector 210 is attached to a surface or coupled to the diaphragm 220 to measure direct movement of the diaphragm surface resulting from an AC current that is applied to the diaphragm 220. Measuring the movement of the diaphragm surface may include a direct or indirect measurement of the membrane or diaphragm using various types of oscillation amplitude detectors, as discussed above. The diaphragm 220 is placed in an opening in the wall 102 of the chamber 100 (FIG. 1), in the top of the chamber 100, or in the door 101, and sealed with a silicone sealant. To adequately hold the diaphragm or membrane in place, a rubber gasket may be further placed around an edge of the oscillating diaphragm or membrane. The subwoofer or diaphragm 220 (FIG. 2), or one or more surfaces thereof, may be stiff or rigid, so positional movement is proportional to volume changes that occur in the chamber (e.g., chamber 100 of FIG. 1). One or more wires 230 electrically connect the oscillation amplitude detector 210 to the display unit or computer 105 and/or to an external unit. The wire(s) 230 may be connected to the oscillation amplitude sensor or detector 210 in a manner allowing them to vibrate or oscillate along with the diaphragm 220. In various embodiments of the present invention, the oscillation amplitude sensor or detector 210 measures the amplitude of the oscillation of the diaphragm using an accelerometer. Although the accelerometer may not provide a direct positional measurement, it provides the amplitude of the oscillation (i.e., the difference between highest and lowest, or outermost and innermost, positions of the oscillating diaphragm 220, assuming the membrane/diaphragm oscillates at a fixed frequency). This is because there is a direct relationship between an acceleration amplitude and oscillation or position amplitude for a fixed frequency signal.

Alternatively, the diaphragm 220 (e.g., a commercially available, off-the-shelf subwoofer speaker) may have two separate coils (e.g., dual coils). A first coil can drive or power vibration or oscillation of the diaphragm or speaker, and a second coil can generate a voltage induced by an oscillating magnet on the diaphragm or speaker. The voltage on the second coil may be proportional to a velocity of the diaphragm 220 or magnet thereon. At a fixed frequency, there is a direct relationship between the amplitude of a velocity signal and the amplitude of a displacement signal. As a result, the second coil of the speaker may be used to determine the amplitude of the oscillation. Consequently, velocity and displacement signals may be used to increase the accuracy of the body fat measurement system. Conventional measuring apparatuses required a second chamber to provide any feedback mechanism (besides creating a second chamber) to the oscillation amplitude.

An Exemplary Method and Software Program for Determining a Volume of an Object in a Single Chamber Volume Measurement Apparatus

A further aspect of the invention relates to a method of determining a volume of a person, animal or object in a single chamber volume measuring apparatus, including measuring a first position amplitude of an oscillating membrane or diaphragm in the apparatus without the person, animal, or object therein, placing the person, animal, or object in the apparatus, measuring a second position amplitude of the oscillating membrane or diaphragm with the person, animal, or object therein, and calculating the volume of the person, animal, or object from the first and second position amplitudes. The chamber has a volume and/or size that is sufficient to enclose the person, animal or object. The temporal order of measurement between the “first” measurement described above and “second” measurement may be interchanged.

Measurement and calibration using Boyle's law under adiabatic conditions may be determined using the following equation:

dP/P=(dV/V)^{γ} (1)

where γ is equal to 1.4 for air. When dP/P is much smaller than 1, the equation may be accurately approximated by the following equation:

dV/V=(1/γ)*dP/P or V=dV/dP*(γ/P) (2)

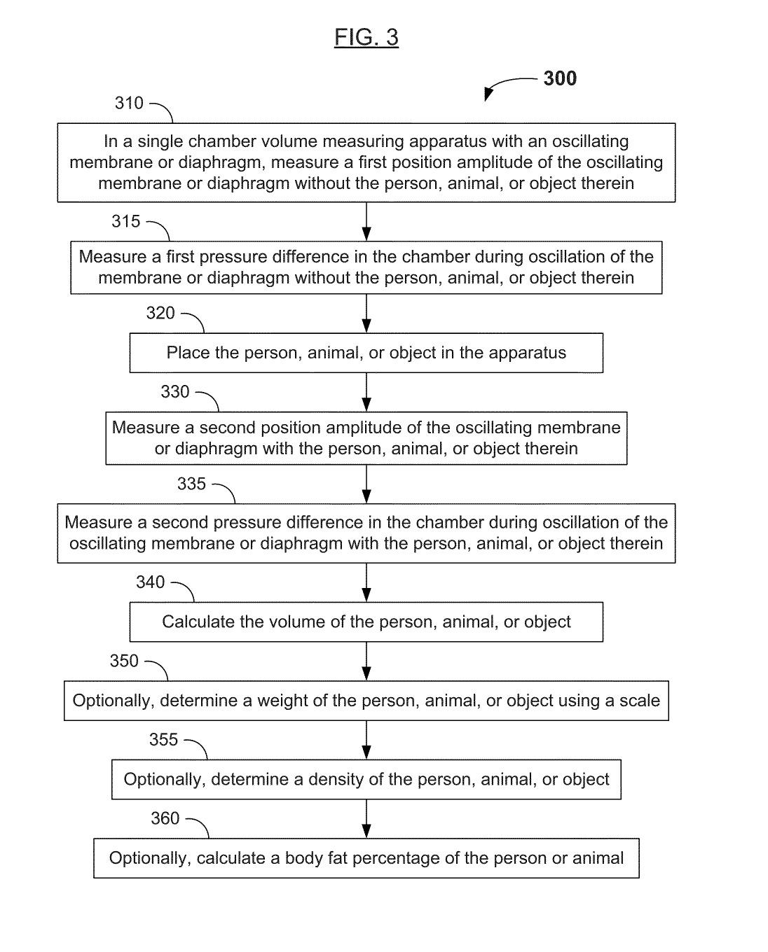

FIG. 3 shows a flow chart for an exemplary method 300 of determining a volume and/or density of a person, animal or object, and/or a body fat percentage of a person or animal. In various embodiments of the present invention, an oscillation amplitude detector (e.g., an oscillation amplitude or position sensing element) is used to measure dV. Generally, the oscillation amplitude detector does not need to be calibrated for absolute movement. However, the oscillation amplitude detector may be calibrated to maintain a linear relationship between the response of the sensor and the movement of the diaphragm surface resulting from an AC current that is applied to the diaphragm, as calculated according to the following equation:

dV=A*k*dx (3)

where dV equals the change in chamber volume, k equals a constant (unknown or empirically determinable for a given apparatus and diaphragm), dx equals the oscillation amplitude of the diaphragm, and A equals the surface area of the diaphragm. A rigid membrane or diaphragm may facilitate the desired linear relationship.

At 310, a measurement is made with an empty chamber to determine a first position (or oscillation) amplitude of the oscillating membrane or diaphragm, using the oscillation amplitude detector or position sensing element as described herein. At 315, a pressure difference measurement is made with the empty chamber to determine the pressure change amplitude of the sensor with the empty chamber, represented by dPe in Equation (4) below. The volume of air in the empty chamber (Ve) is calculated by the following equation:

Ve=A*k*dxe/dPe*(b 1/γ*Patm) (4)

where dxe and dPe are respective oscillation and pressure amplitudes of the system with the empty chamber. Patm equals the ambient average pressure or atmospheric pressure. Thus, the volume of air in the empty chamber may be calculated or calibrated, if it is not already known.

At 320, the person, animal or object is placed in the chamber, and a second position amplitude of the oscillating membrane or diaphragm is measured with the person, animal, or object therein at 330, and a second pressure difference in the chamber during oscillation of the oscillating membrane or diaphragm with the person, animal, or object therein at 335. The volume of air in the chamber when the object or subject is inside the chamber (Vs) is determined by the following equation:

Vs=A*k*dxs/dPs*(1/γ*Patm) (5)

where dxs and dPs are respective oscillation and pressure amplitudes of the system with the subject or object inside the chamber. Dividing the volume of air with and without the object or subject inside the chamber is determined by the following equation:

Vs/Ve=(dPe/dxe)/(dPs/dxs) (6)

Thus, the ratio of the empty chamber volume (Ve) to the air volume remaining in the chamber with the subject therein (Vs) is given or determined by the ratio of the pressure changes divided by the ratio of the oscillation amplitudes. It is noted that the empty chamber volume (Ve) and the air volume remaining in the chamber with the subject therein (Vs) can be determined in any sequence (e.g., the position amplitude of the oscillating membrane or diaphragm and the pressure difference can be measured with the person, animal, or object in the chamber before measuring the position amplitude of the oscillating membrane or diaphragm and the pressure difference without the person, animal, or object in the chamber).

Typically, the empty chamber volume may be calibrated either during manufacturing or with a reference volume test. Thus, the final subject or object volume is determined by the following equation:

V_{final}=Ve−Vs=Ve*(1−[dPxe/dPxs]) (7)

where dPxe equals dPe/dxe and dPxs equals dPs/dxs, the normalized pressure amplitudes that are normalized by the amplitude of the oscillation amplitude detector. Ve is approximately equal to the empty volume of the chamber, but can be calibrated in manufacturing or using a reference object of known volume. Ve may therefore be a calibration constant, and is generally slightly larger than the true geometric volume of the chamber due to imperfect chamber stiffness or imperfect sealing of the chamber. Consequently, at 340, the volume of a body or object can be measured in the single chamber apparatus using equation (7) above.

In an exemplary embodiment of the present invention, the empty chamber has a volume of 1000 liters. A diaphragm (e.g., a commercially available, off-the-shelf 10″ subwoofer speaker) is mounted in a 10″ circular opening in the wall of the chamber, in which a front portion of the speaker (e.g., speaker face or faceplate) faces the inside the chamber, and a rear portion of the speaker (e.g., including the spider) is outside of the chamber. The 10″ subwoofer has a surface area of 500 cm². Modulating the speaker at a frequency of 10 Hz and at an oscillation amplitude of 1 cm peak to peak results in a pressure wave inside the chamber at 10 Hz. The pressure wave is sensed by the difference in the oscillation of the diaphragm indicated by the oscillation amplitude sensing element. The total volume change peak to peak of the speaker movement is then 0.5 L. The pressure wave peak to peak value inside the chamber is determined by the following equation:

dP=Patm*1.4*dV/V (8)

Using typical values for atmospheric pressure, the peak to peak pressure wave (or pressure amplitude or difference) is 70 Pa. The exact amplitude of the speaker movement may vary with temperature and time, but the results generally do not. An exact measurement simultaneously between the pressure wave amplitude and speaker displacement amplitude is performed. The two pressures divided by each other equals dPxe. Subsequently, the process is repeated with the subject inside the chamber. The presence of the subject inside the chamber will modify the amplitude of the speaker movement relative to the empty chamber. Another simultaneous measurement is performed between the speaker displacement (the oscillation or position amplitude) and the pressure wave amplitude, equaling dPxs. Alternatively, the measurements can be taken in the opposite sequence (simultaneous measurement of the pressure wave amplitude and speaker displacement amplitude with the subject inside the chamber first, then with the chamber empty afterwards). The volume of the subject is determined by the following equation:

V_{subject}=V_{chamber}*(1−[dPxe/dPxs]) (9)

For example, when dPxe equals 70 Pa/cm, and dPxs equals 75 Pa/cm (when the subject is in the chamber), the volume of the subject is 1000*(1−[70/75])=66.7 L. Once the volume of the subject is determined, the weight of the subject can be determined at 350 using a conventional weight scale. Subsequently, the density of the subject is determined at 355 by dividing the weight of the subject by the volume of the subject. Thus, if a subject weighs 70 kg, the density of the subject would be 1.0495 g/cm³. An empirical formula (e.g., the Siri formula) is used at 360 to translate the subject's density to the subject's body fat, for example using the following equation:

Body Fat %=100*([4.95/density]−4.5) (10)

Using equation (10), the subject has 21.6% body fat.

Depending on the frequency of the pressure wave in the chamber, the pressure wave may enter the subject's lungs. However, a correction factor for pressure waves that enter the subject's lungs may be included in the calculations.

An Exemplary Method of Making a Single Chamber Volume Measuring Apparatus

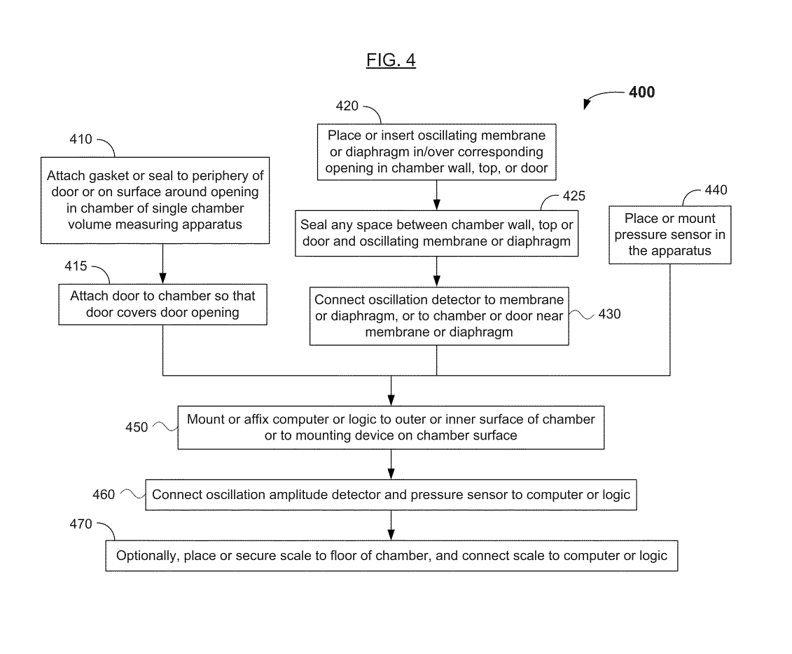

The present invention further relates to method of making a single chamber volume measuring apparatus. A flow chart 400 for an exemplary method is shown in FIG. 4. The method comprises attaching a door to a chamber (e.g., at 415), mounting or positioning an oscillating membrane in a wall, top, or door of the chamber (e.g., at 420), placing or mounting a pressure sensor/transducer on an internal surface of the chamber or door (e.g., at 440), and connecting an oscillation amplitude (e.g., position sensing) element or detector on the oscillating membrane (e.g., at 430). An opening is provided in the wall, top or door of the chamber to place or insert the oscillating membrane. After the oscillating membrane is placed into the opening, a silicone sealant or otherwise similar product is placed around the oscillating membrane to provide an airtight seal (e.g., at 425). Alternatively, a gasket may be inserted around the opening in the chamber wall, top, or door and attached to a periphery of the membrane, in no particular order, to seal any space between the oscillating membrane and the chamber.

In exemplary embodiments of the present invention, the chamber has rigid and/or transparent walls, and the door is hingedly affixed thereto. Generally, the chamber size has a volume that is sufficient to enclose the person, animal or object, and is substantially constant when the door is closed and the pressure inside the chamber changes. In some embodiments, one or more magnetic strips may be affixed to either (i) the stationary part of the chamber, around the door opening on the surface facing towards the door, or (ii) the peripheral surface of the door facing towards the stationary part of the chamber. In such embodiments, one or more complementary or matching magnetic or ferromagnetic metal strips may be placed or affixed around the other of the stationary part of the chamber or the door, or the surface of the door or the stationary part of the chamber that faces the other may be made of a ferromagnetic metal. The magnetic strips generally have a magnetic strength sufficient to keep the door closed when the pressure changes or fluctuates inside the chamber during a volume measurement. A gasket or seal may be placed or formed around the periphery of the door and/or the door opening in the chamber (e.g., at 410) to make the chamber airtight when the door is closed.

In various embodiments of the present invention, the oscillation amplitude detector responds proportionally to the displacement of the membrane. Wires may be connected between the oscillation amplitude detector and a computer and/or operating logic for the system (e.g., at 460). Alternatively, the oscillation amplitude detector or position sensor may send membrane displacement information to the computer wirelessly. The computer and/or logic and a display unit connected thereto are mounted or connected to a surface or a mounting device on the inside and/or outside of the chamber (e.g., at 450).

This method may also further comprise programming a memory in the logic and/or computer with a set of instructions to: (i) measure a first position amplitude of an oscillating member or diaphragm before and the subject or object enters the chamber, (ii) place the subject or object in the chamber, (iii) seal the chamber, (iv) measure a second position amplitude of the oscillating membrane or diaphragm with the subject or object in the chamber, and/or (v) determine a displaced volume of air in the chamber from the chamber volume and the first and second position amplitudes. Various functions and data may be stored in the memory of the computer or logic (e.g., control system).

In other embodiments of the present invention, the method of making may further comprise forming or attaching a seal or molding on a surface of at least one of the door and the chamber, wherein the seal or molding is configured to make the chamber airtight when the door is closed and the pressure inside the chamber changes. Generally, the surface on which the seal or molding is formed or attached faces an opposing surface of the other of the door and the chamber. For example, when the door opens to the outside, the seal or molding is formed on or attached to an outer surface of the chamber (e.g., in a region that overlaps with a peripheral portion of the door), or the peripheral portion of the inner surface of the door. When the door opens to the inside, the seal or molding is formed on or attached to an inner surface of the chamber (e.g., in a region that overlaps with a peripheral portion of the door) or the peripheral portion of the outer surface of the door.

Various embodiments of the present method further comprise forming or attaching a handle on an external and/or an internal surface of the chamber. Additionally, a weight scale may be placed on or attached or affixed to a bottom internal portion of the chamber (e.g., at 470), and the scale may be connected (e.g., wirelessly or through one or more wires) to the computer or logic.

The method of making can further include attaching, affixing and/or forming other components described herein to, on or in the system, in various ways consistent with the present disclosure and/or the knowledge in the art.

### CONCLUSION/SUMMARY

The foregoing descriptions of embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed is:

1. A system or apparatus, comprising:
a) a chamber having a door thereon or affixed thereto, and a fixed volume therein when the door is closed;
b) an oscillating membrane or diaphragm in a wall of the chamber or door;
c) a pressure sensor and/or a pressure transducer configured to measure pressure fluctuations in the chamber; and
d) an oscillation amplitude detector coupled to the oscillating membrane or diaphragm.

2. The system or apparatus of claim 1, further comprising a computer or logic configured to determine a value proportional to the pressure amplitude in the chamber resulting from oscillations of the oscillating membrane or diaphragm and a value proportional to the volume displacement from oscillation or position amplitudes of the oscillating membrane or diaphragm with and without a subject or object in the chamber.

3. The system or apparatus of claim 1, wherein the chamber has a volume sufficient to enclose a person, animal, or object therein, and the system or apparatus is configured to measure a volume of the person, animal, or object.

4. The system or apparatus of claim 1, wherein the chamber comprises one or more transparent walls.

5. The system or apparatus of claim 1, wherein the chamber comprises (i) a first section comprising the door and (ii) a second, stationary section configured to house or contain a subject or object.

6. The system or apparatus of claim 3, wherein the volume of the chamber does not vary with relatively small pressure changes or fluctuations.

7. The system or apparatus of claim 1, wherein the oscillating membrane or diaphragm comprises a rigid oscillating membrane or diaphragm.

8. The system or apparatus of claim 1, wherein the oscillating membrane or diaphragm oscillates at a frequency of from 1 to 100 Hz.

9. A system or apparatus of claim 1, wherein the oscillating membrane or diaphragm has an area of from 10 to 5000 cm².

10. A system or apparatus of claim 1, wherein the oscillation amplitude detector comprises (i) an optical light source and a light detector or (ii) an accelerometer.

11. A system or apparatus of claim 1, wherein the oscillation amplitude detector is configured to measure movement and/or displacement of the oscillating membrane or diaphragm.

12. A system or apparatus of claim 1, wherein the system or apparatus is configured to calculate a percentage or other proportion of body fat of a person and/or animal.

13. The system or apparatus of claim 2, wherein the computer or logic is on the exterior of the apparatus, and is configured to control operations of the apparatus and perform calculations to determine body volume, body fat content, and/or weight of a person or animal.

14. A method of determining a volume of a person, animal, or object in a single chamber volume measuring apparatus, comprising:
a) measuring a first oscillation or position amplitude of an oscillating membrane or diaphragm in the apparatus without the person, animal, or object therein;
b) placing the person, animal, or object in the apparatus;
c) measuring a second oscillation or position amplitude of the oscillating membrane or diaphragm with the person, animal, or object therein; and
d) calculating the volume of the person, animal, or object.

15. The method of claim 14, further comprising calculating a body fat percentage of the person or animal.

16. The method of claim 14, further comprising measuring a first pressure difference in the single chamber during oscillation of the oscillating membrane or diaphragm without the person, animal, or object therein, and measuring a second pressure difference in the single chamber during oscillation of the oscillating membrane or diaphragm with the person, animal, or object therein.

17. The method of claim 16, further comprising determining a first volume of air in the single chamber without the person, animal, or object therein, and determining a second volume of air in the single chamber with the person, animal, or object therein.

18. The method of claim 17, further comprising determining a weight of the person, animal, or object using a weight scale, and determining a density of the person, animal, or object by dividing the weight of the person, animal, or object by the volume of the person, animal, or object.

19. The method of claim 18, further comprising converting the density of the person, animal, or object to a body fat percentage.

20. The method of claim 14, wherein the first oscillation or position amplitude of the oscillating membrane or diaphragm is measured before the second oscillation or position amplitude.

21. A method of manufacturing a system or apparatus to measure a volume of a person, animal, or object, or body fat percentage of the person or animal, comprising:
a) attaching door to a chamber;
b) mounting or positioning an oscillating membrane or diaphragm in a wall of the chamber;
c) placing a pressure sensor/transducer on an internal surface of the chamber; and
d) coupling an oscillation amplitude detector to a surface of the oscillating membrane or diaphragm.

22. The method of claim 21, wherein the chamber has rigid walls, and one or more of the walls and/or the door are transparent.

23. The method of claim 21, wherein the chamber comprises a stationary section configured to house, contain, or hold the person, animal, or object, and the chamber has a volume sufficient to enclose the person, animal or object.

24. The method of claim 21, further comprising mounting or connecting a computer or operational logic to the apparatus.

25. The method of claim 24, further comprising programming a memory in the computer or operational logic with a set of instructions to (i) measure a pressure difference during oscillations of the oscillating membrane or diaphragm before and after the person, animal, or object enters the chamber, (ii) determine a ratio of oscillation amplitudes of the oscillating membrane or diaphragm before and after the person, animal, or object enters the chamber, and (iii) determine a volume of air in the chamber displaced by the person, animal, or object using the oscillation amplitudes and pressure differences.
